(19) United States
(12) Patent Application Publication
(10) Pub. No.: US 2015/0329445 A1
(43) Pub. Date: Nov. 19, 2015

(54) PROCESS FOR RECOVERING HYDROCARBONS FROM POLYOLEFIN PLANTS AND APPARATUS SUITABLE FOR THIS PURPOSE

(71) Applicant: THYSSENKRUPP UHDE GMBH, Dortmund (DE)

(72) Inventors: Michael Kleiber, Hattersheim (DE); Tobias Friederich, Frankfurt am Main (DE)

(73) Assignee: THYSSENKRUPP INDUSTRIAL SOLUTIONS AG, Essen (DE)

(21) Appl. No.: 14/381,783

(22) PCT Filed: Feb. 9, 2013
(86) PCT No.: PCT/EP2013/000400

§ 371 Date: Aug. 28, 2014

(30) Foreign Application Priority Data
Feb. 28, 2012 (DE) 102012003741.4

(51) Int. Cl. C07C 7/09; F25J 3/06; C08F 110/08; C08F 110/14; C08F 110/02; C08F 110/06

(52) U.S. Cl. CPC C07C 7/09; C08F 110/02; C08F 110/06; C08F 110/08; C08F 110/14; F25J 3/061

(57) ABSTRACT

Disclosed herein is a method and apparatus for recovering hydrocarbons from polyolefin plants. A method of the present disclosure includes introducing a hydrocarbon-containing inert gas from a residual monomer separation unit of a polyolefin plant into a condensation and separation device, condensing hydrocarbons from a hydrogen-containing inert gas in the condensation and separation device, separating the condensed inert gas into a condensed hydrocarbon-containing product and purified inert gas in the device, and sending the condensed hydrocarbon-containing product to a downstream further separation device for removal of dissolved gasses therefrom. A device of the present disclosure includes a condensation and separation device for condensing hydrocarbons from an inert gas and separating condensed hydrocarbon-containing inert gas into a condensed hydrocarbon-containing product and a purified inert gas, and a further separation device connected downstream for purifying the condensed hydrocarbon-containing product originating from the condensation and separation device by separating off dissolved gases.

The invention relates to a process for recovering hydrocarbons, in particular residual monomers, from polyolefin plants, and also a device suitable therefor.

In most polyolefin plants, the polymer produced, after it has been discharged from the reactor and expanded, is freed using nitrogen and/or steam from unreacted monomer (hereinafter “residual monomer”) and other hydrocarbons having a low carbon number. In current polyolefin plants, this takes place in a degassing device in which nitrogen and/or steam are passed through in counterflow to the polymer.

The resultant off-gas stream generally also contains, in addition to nitrogen and steam, valuable monomer, for example ethylene, propylene, butylene or hexene, and also other hydrocarbons, for example ethane, propane or butane. In many cases, this off-gas stream is passed into an incineration or flare.

In the prior art there are already proposals for recovering residual monomers in polyolefin production or for separating off low alkanes or alkenes from gas mixtures which originate from cracking processes or are refinery gases.

EP 1 160 000 A1 discloses a process for recovering nitrogen and/or propylene in the production of polypropylene. The process comprises separating off propylene by means of nitrogen in a degassing device, isothermal compression of the gas mixture and separating off the propylene from nitrogen from the compressed gas mixture by means of a membrane.

EP 1 148 309 A1 discloses an improved process for separating a gas mixture which, in addition to hydrogen, contains ethane, ethylene, propane and propylene. The gas mixture originates from a thermal cracking process and the individual components are separated off by cooling and separating off liquefied components. The process is very energy efficient and is characterized in that a part of the cooling proceeds using a cold cooling stream which is generated by cold expansion of a pressurized cooling stream. In this process the cold-expanded pressurized cooling stream is a gaseous cooling stream which was produced in a gas expansion cooling process having a closed circuit.

In DE 10 2004 061 772 A1, a process for producing propene from propane is described. In this process, a product stream containing steam, nitrogen, carbon oxides, hydrogen and various lower alkanes and alkenes is formed. This stream is purified by condensation of some of the steam. The non-condensable or low-boiling gas components are removed by contact with an inert adsorbent, and the remaining gas is partially condensed by cooling and product streams are obtained which principally contain ethane and ethene, and also propane and propene. These product streams are then separated into their individual components by distillation.

Finally, GB-A-1,069,981 discloses a process for separating a gas mixture in which the cooling potential of liquefied natural gas is utilized. The gas mixture that is to be separated originates from the off-gas of a refinery and, in addition to nitrogen, hydrogen and carbon monoxide, principally contains low alkanes and alkenes. The process comprises an at least two-stage cooling of the gas mixture and a fractional condensation and separation of components present therein.

DE 36 26 884 A1 describes generally a process for separating off hydrocarbons from a gas mixture. Here, reference is made to gasoline-comprising exhaust air during handling of fuels, and also to solvent-comprising vapors from papermaking. This document also describes a condensation and separation device in which heat recovery measures are carried out. However, in this document, no external interconnection is disclosed a component of which is such a condensation and separation device and which would be suitable for recovering from an off-gas stream a monomer in accordance with the requirements of polyolefin production.

In DE 10 2008 024 427 A1, a process is described in which the gas that is to be treated is cooled in a condenser in direct contact with a coolant. Indirect contact between coolant and the gas that is to be treated is not disclosed. It is doubtful that, in the process disclosed in this document, the vaporized coolant can be fed into a works grid, since owing to the direct contact with the gas that is to be treated, in the case of an operating fault, the possibility of contamination of the works grid with the hydrocarbon exists.

To date, for recovering hydrocarbons from the off-gas stream of polyolefin plants in practice, principally membrane systems are employed which are burdened with various disadvantages, inter alia with relatively high operating and maintenance costs.

The object of the present invention is providing an improved process and a plant suitable therefor for recovering hydrocarbons in polyolefin production. The process and the plant are distinguished by a high separation efficiency with simultaneously low energy consumption and thereby permit an economic recovery of residual monomers and other low hydrocarbons in polyolefin production.

An improved separation of the off-gas stream from the degassing device into hydrocarbons and nitrogen would permit the separated components to be reused.

Unreacted monomer or other hydrocarbons can be fed, for example, back to the polymerization reactor, to another consumer, or recirculated to further separation (e.g. in a cracker). As a result, monomers and other hydrocarbons can be almost completely recovered or further processed.

The nitrogen stream also could, if recovered in clean enough condition, be at least in part reused in the process, e.g. in the degassing device.

Via recovery of the substreams, the economic efficiency of the polyolefin plant will be able to be considerably increased.

The invention relates to a process for recovering hydrocarbons from a plant for producing polyolefins which comprises the measures:
i) introducing a hydrocarbon-containing inert gas (9) from a residual monomer separation (20) of a polyolefin plant into a condensation and separation device (1),
ii) introducing liquid nitrogen into the condensation and separation device (1),
iii) condensing at least a part of the hydrocarbons from the hydrocarbon-containing inert gas (9) in the condensation and separation device (1), utilizing the energy of vaporization of the liquid nitrogen (10),
iv) separating the condensed hydrocarbon-containing inert gas into a condensed hydrocarbon-containing product (12) and also purified inert gas (14) in the condensation and separation device (1), and
v) introducing the condensed hydrocarbon-containing product (12) from the condensation and separation device (1) into a downstream further separation device (16) in which dissolved gases from the condensed hydrocarbon-containing product (12) are separated off.

In addition, the invention relates to a device for recovering hydrocarbons from a plant for producing polyolefins which comprises at least the elements:
A) condensation and separation device (1) for condensing hydrocarbons from an inert gas and for separating the condensed hydrocarbon-containing inert gas into a condensed hydrocarbon-containing product (12) and a purified inert gas (14),
B) connection line for the hydrocarbon-containing inert gas (9) between a residual monomer separation (20) of a polyolefin plant and the condensation and separation device (1),
C) line for the introduction of liquid nitrogen (10) into the condensation and separation device (1),
D) lines for removing the condensed hydrocarbon-containing product (12), the purified inert gas (14) and the vaporized nitrogen (11) from the condensation and separation device (1), and
E) a further separation device (16) which is connected downstream of the condensation and separation device (1) and which serves for purifying the condensed hydrocarbon-containing product (12) that originates from the condensation and separation device (1) by separating off dissolved gases, preferably nitrogen.

The plant according to the invention or the process according to the invention uses the cold from liquid nitrogen in order to precipitate the condensable fractions of the off-gas stream and to separate these from the non-condensable components.

The hydrocarbons separated off by the process according to the invention are generally non-polymerized alkenes and optionally alkanes having two to ten carbon atoms which are formed in the polymerization or which are present as components in the feed stream. Preferably the hydrocarbons separated off are propylene and optionally propane, or ethylene and optionally ethane, and also a mixture of these hydrocarbons. Depending on the type of polyolefin production, higher saturated and unsaturated hydrocarbons can also be present, for example saturated or monounsaturated or polyunsaturated hydrocarbons having four to ten carbon atoms. Examples thereof are alpha-pentene, alpha-hexene, alpha-heptene, alpha-octene, alpha-nonene, alpha-decene, pentane, hexane, heptane, octane, nonane, decane, 1,3-butadiene, isoprene, styrene or alpha-methylstyrene.

The inert gas used in the process according to the invention is generally nitrogen to which small amounts of steam are optionally added.

In a preferred embodiment of the process according to the invention, the gas stream that is to be separated is preferably cooled stepwise in counterflow to the cold streams that have been separated, i.e. to the streams of the cold inert gas and of the cold condensed hydrocarbon-containing product. The use of counterflow heat exchangers in this preferred interconnection considerably reduces the cooling power required for the actual separation step (condensation of the hydrocarbons). In a second step of this preferred process, the gas mixture that is to be separated is then cooled in the actual condensation and separation device (1) by heat exchange with the cooling medium, that is either by indirect heat exchange with vaporizing nitrogen or by indirect heat exchange with low-temperature gaseous nitrogen which is obtained by vaporizing liquid nitrogen. In this process the condensable components of the off-gas stream condense out and are simultaneously deposited on the cold heat exchanger surfaces in the condensation and separation device (1), whereby separation into condensed hydrocarbon-containing product (12) and purified inert gas (14) proceeds. In such low-temperature condensations, the formation of aerosols can occur. This is suppressed by suitable technical measures within the condensation and separation device (1). Likewise, by suitable measures for heat and cold recovery within the condensation and separation device (1), the utilization of the cold of the supplied liquid nitrogen can be optimized. These facts are known to those skilled in the art.

Condensation and separation devices (1) are commercially available. In these systems, the gas that is to be treated, that is to say the condensed hydrocarbon-containing inert gas, is cooled, and the liquid nitrogen used is vaporized and heated by indirect contact between coolant and the gas that is to be treated.

The phase separation between condensed hydrocarbons and inert gas from the off-gas stream takes place in the condensation and separation device (1).

The cold inert gas, preferably nitrogen, can be pressurized by a compressor and recirculated back to the polyolefin plant, preferably the residual monomer separation (20). In a further embodiment, the off-gas stream can already be pressurized before entry into the condensation and separation device (1) or before entry into a drying device (3a, 3b) connected upstream of this condensation and separation device (1) by means of a compressor (2). The purified inert gas (14) from the condensation and separation device (1) can also be released directly, that is to say without further purification, into the atmosphere.

The condensed hydrocarbon-containing fractions of the separated off-gas stream, that is of the product stream, can be pressurized by a pump (6) and can be heated with the abovementioned counterflow heat exchangers against the off-gas stream that is to be separated. In another embodiment, the product stream can also be heated by another heat source.

In the process according to the invention, a further separation device (16) is connected downstream of the condensation and separation device (1). This further separation device (16) preferably comprises a pump, a heat exchanger and a phase separation device, wherein heat exchanger and phase separation device can also be constructed as one functional unit. Via pump and heat exchanger, suitable pressure and temperature conditions are established in order to remove from the product (12) by expansion gases dissolved in the product (12).

The interconnection according to the invention of condensation and separation device (1) to the further separation device (16) is of importance for the recovery of residual monomers from polyolefin production.

By the above described treatment of the condensed hydrocarbon-containing product (12) gases dissolved in the liquid, preferably nitrogen, which were dissolved at the low temperature, outgas in the further separation device (16). The hydrocarbons remain liquid and, via this second purification step, arise in very high purity. The hydrocarbons that are freed from the dissolved gases and purified are collected in the separation device (16), preferably in a separation vessel, and can be recirculated, for example, into the feed stream of the reaction unit (17) of the polyolefin plant or fed to another device, for example a cracker. Alternatively, these purified hydrocarbons can be subjected to a further purification, for example a distillation. The gases separated off in the further separation device (16) can be fed back to the residual monomer removal (20) of the polyolefin plant and thereby at least partly recovered.

Also, the inert gas stream separated off in the condensation and separation device (1) and the gas stream from the vaporization of the liquid nitrogen can be used for precooling the off-gas stream that is to be purified and these streams can be introduced into the polyolefin plant. In this process the gas stream from the vaporization of the liquid nitrogen is used as protective gas, and the inert gas stream separated off is recirculated at least in part to the residual monomer removal (20).

Owing to the use of very low temperatures and the separation of condensable and non-condensable fractions, a very high separation sharpness is achieved which permits reuse of both components.

Depending on the application, additional components, such as separation of the hydrocarbons by distillation or drying of the feed stream, can be added.

Since the plant preferably succeeds without compressors, the process is distinguished by a very low energy consumption and low operating costs.

In a preferred variant of the process according to the invention, the product (12, 13), after it is removed from the further separation device (16), is fed to a distillation or separation by desorption, and can then be introduced, for example, into a cracker or into a plant for dehydrogenation of olefins, such as propane, or the product (12, 13), after it is removed from the further separation device (16), is fed to another chemical reaction, which can further use the hydrocarbons directly.

In a further preferred variant of the process according to the invention at least one dryer (3a, 3b) and/or at least one compressor (2) are situated between the residual monomer separation (20) and the condensation and separation device (1).

Advantages of the process according to the invention/plant according to the invention which may be mentioned are:
virtually complete recovery of the components of the off-gas stream and possible return of the components as valuable materials;
dispensing with compressors leads to a very much lower electrical power requirement than with other recovery processes;
the use of available liquid nitrogen as cold source avoids the energy-intensive and expensive cooling with a refrigeration plant or by vaporization of a product substream;
simple structure of the plant having a minimum of moving parts leads to low capital costs and low maintenance costs;
the vaporized nitrogen can be further used as protective gas; this gives a cost advantage, since vaporization of the nitrogen simultaneously serves for condensation of the hydrocarbons in the condensation and separation device.

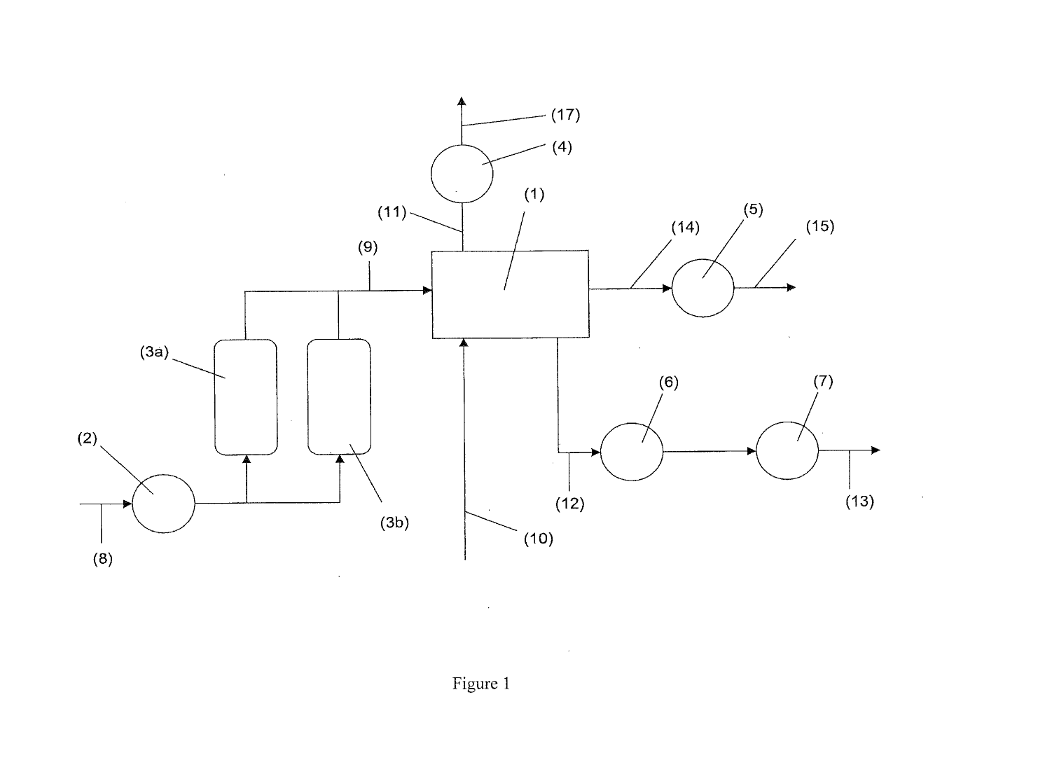

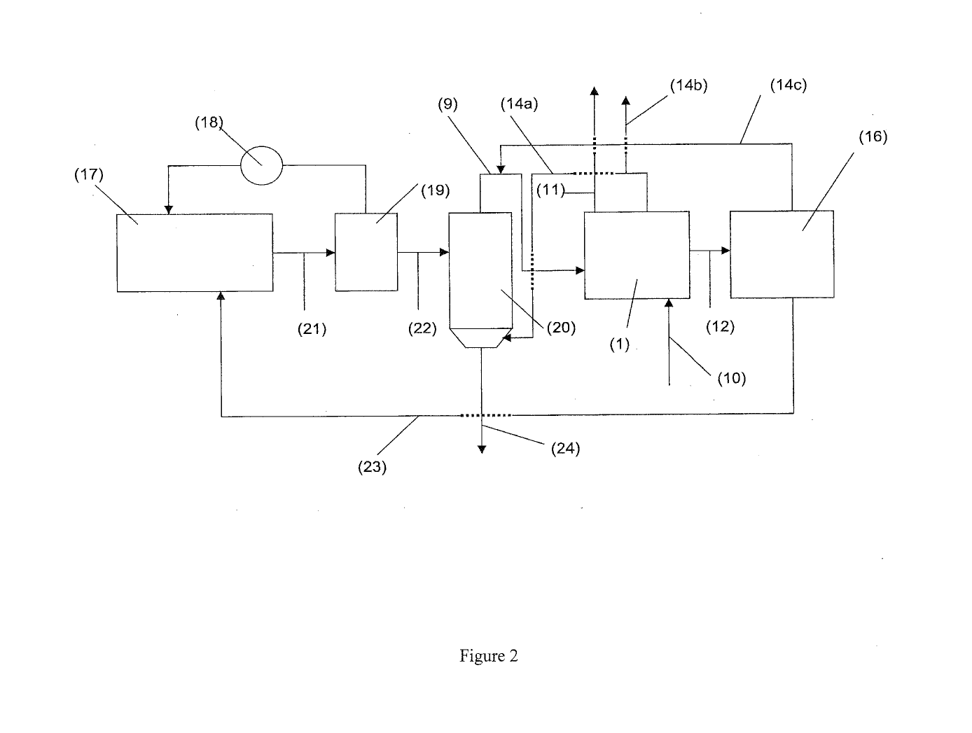

FIGS. 1 to 2 describe by way of example and schematically the process according to the invention and the device according to the invention and also incorporation thereof into a polyolefin plant.

In FIG. 1, a variant of the process according to the invention and of the plant according to the invention for the recovery of hydrocarbons is shown. A condensation and separation device (1) is shown which is connected to dryers (3a, 3b), compressor (2), pump (6) and heat exchangers (4, 5, 7). The inert gas stream (8) containing alkenes and optionally alkanes and which is from a residual monomer removal which is not shown is passed with the aid of the compressor (2) into dryers (3a, 3b) and is introduced into the condensation and separation device (1) as a dried inert gas stream (9) containing alkenes and optionally alkanes. In addition, a stream of liquid nitrogen (10) is introduced into the condensation and separation device (1). In the condensation and separation device (1), the alkenes, and the optionally present alkanes are condensed with simultaneous vaporization of the liquid nitrogen. The vaporized liquid nitrogen (11) leaves the condensation and separation device (1) and passes through heat exchanger (4) in which the vaporized but still cold nitrogen is further heated, and leaves the plant as nitrogen stream (17) which is used, e.g. as protective gas, in the polyolefin plant which is not shown. In the condensation and separation device (1), in parallel to the condensation of the condensable components, the inert gas stream containing condensed hydrocarbons is separated into condensed hydrocarbon-containing product (12) and purified inert gas (14), preferably nitrogen. The stream of the product (12) is ejected from the condensation and separation device (1) and conveyed by means of a pump (6) into a heat exchanger (7) in which the condensed and still cold product (12) is heated. The product (12) leaves the plant as heated product stream (13), is introduced into a further separation device (16), which is not shown, for removal of dissolved gases and can, e.g., after leaving the further separation device (16), be added to the feed stream of the polyolefin plant or be subjected to a further separation into individual components. The purified inert gas (14) separated in the condensation and separation device (1) is ejected and passed into a heat exchanger (5) in which the still cold inert gas (14) is heated, preferably to ambient temperature. The inert gas (14) leaves the plant as heated inert gas stream (15) and can, e.g., be fed to the residual monomer removal in the polyolefin plant. Alternatively, the inert gas stream (15) can also be released directly into the surroundings.

FIG. 2 shows an interconnection of a plant according to the invention operated in two stages for recovering hydrocarbons, with a polyolefin plant. The figure shows a condensation and separation device (1) which is interconnected via a line to a residual monomer removal (20) of a polyolefin plant. Via this line, a nitrogen stream containing alkenes and optionally alkanes is fed to the condensation and separation device (1). In addition, a stream of liquid nitrogen (10) is fed to the condensation and separation device (1), which stream of liquid nitrogen is vaporized in the condensation and separation device (1) and leaves this as nitrogen stream (11). The condensed hydrocarbon-containing product stream (12) is introduced via a line into a further separation device (16), where owing to suitable pressure and temperature conditions, the product (12) is freed from the dissolved nitrogen and thereby product (12) is separated into purified condensed hydrocarbons (23) and heated nitrogen (14c) containing hydrocarbons. The heated nitrogen (14c) that is separated off leaves the further separation device (16) and can be combined with the inert gas stream (9) that originates from the residual monomer removal (20) of the polyolefin plant and is loaded with residual monomer. The nitrogen stream removed from the condensation and separation device (1) can be fed further as stream (14a) to the residual monomer removal (20) of the polyolefin plant and/or be ejected from the plant as stream (14b) and released directly into the surroundings. The purified condensed hydrocarbons (23) separated off in the further separation device (16) are removed from the further separation device (16) and fed back to the reaction unit (17) of the polyolefin plant or ejected (which is not shown) from the plant and fed to other uses. The polyolefin plant which is shown schematically in FIG. 2 consists of the reaction unit (17) and a polymer separation (19) which is connected to the reaction unit (17) via line (21), and also a residual monomer removal (20) which is connected to the polymer separation (19) via line (22) and a compressor (18) with which monomer which is outgassed from the polymer separation (19) is returned to the reaction unit (17). The purified polymer leaves the residual monomer removal (20) as product stream (24).

1.-16. (canceled)

17. A method of recovering hydrocarbons from a polyolefin manufacturing plant, comprising:
i) introducing into a condensation and separation device a hydrocarbon-containing inert gas from a residual monomer separation of a polyolefin manufacturing plant, wherein the hydrocarbons are at least one of propylene, propylene and propane, ethylene, or ethylene and ethane, and wherein the inert gas is nitrogen;
ii) introducing liquid nitrogen into the condensation and separation device for vaporization therein;
iii) in the condensation and separation device, vaporizing the liquid nitrogen therein to condense at least a part of the hydrocarbons in the hydrocarbon-containing inert gas;
iv) in the condensation and separation device, separating the condensed hydrocarbon-containing inert gas into a condensed hydrocarbon-containing product and a purified inert gas; and
v) introducing the condensed hydrocarbon-containing product from the condensation and separation device into a downstream further separation device; and
vi) separating off dissolved gases from the condensed hydrocarbon-containing product in the downstream further separation device.

18. The process of claim 17, further comprising:
stepwise cooling the hydrocarbon-containing inert gas stream, introduced from the residual monomer separation, in a flow direction counter to the flow direction of at least one of the separated streams of cold condensed hydrocarbon-containing product and cold purified inert gas;
further cooling the hydrocarbon-containing inert gas stream by vaporizing said introduced liquid nitrogen; and
separating off condensable components of the cooled hydrocarbon-containing inert gas from remaining inert gas.

19. The process of claim 17, wherein the dissolved gasses separated off from the condensed hydrocarbon-containing product in the downstream further separation device include nitrogen.

20. The process of claim 17, wherein the downstream further separation device includes a pump, a first heat exchanger and a phase separation device.

21. The process of claim 17, further comprising:
by a first heat exchanger, heating the purified inert gas and generating a cold heat exchanger medium; and
by the cold heat exchanger medium, cooling the hydrocarbon-containing inert gas that is to be separated.

22. The process of claim 21, wherein the first heat exchanger is a counterflow heat exchanger configured to heat the purified inert gas by counterflow cooling the hydrocarbon-containing inert gas that is to be separated.

23. The process of claim 17, further comprising:
by a second heat exchanger, heating the nitrogen vaporized in the condensation and separation device and generating a cold heat exchanger medium; and
directing the cold heat exchanger medium back to the condensation and separation device to reinforce the condensation of the hydrocarbons contained therein.

24. The process of claim 23, wherein the second heat exchanger is a counterflow heat exchanger configured to heat the vaporized nitrogen by counterflow cooling the hydrocarbon-containing inert gas that is to be separated.

25. The process of claim 17, further comprising:
by a third heat exchanger, heating the condensed hydrocarbon-containing product and generating a cold heat exchanger medium; and
directing the cold heat exchanger medium back to the condensation and separation device to reinforce the condensation of the hydrocarbons contained therein.

26. The process of claim 25, wherein the third heat exchanger is a counterflow heat exchanger configured to heat the condensed hydrocarbon-containing product by counterflow cooling the hydrocarbon-containing inert gas that is to be separated.

27. The process of claim 17, further comprising feeding the condensed hydrocarbon-containing product that has passed through the downstream further separation device back to a feed stream for a reaction unit for the polyolefin manufacturing plant.

28. An apparatus for recovering hydrocarbons from a manufacturing plant for producing polyolefins, comprising:
a condensation and separation device configured to both condense hydrocarbons from an inert gas and separate condensed hydrocarbon-containing inert gas into a condensed hydrocarbon-containing product and a purified inert gas;
a first inlet line in communication with the condensation and separation device and configured to permit passage of hydrocarbon-containing inert gas between a residual monomer separation unit of the polyolefin manufacturing plant and the condensation and separation device;
a second inlet line in communication with the condensation and separation device and configured to permit the introduction of liquid nitrogen into the condensation and separation device for vaporization of the liquid nitrogen passing therethrough;
a plurality of outlet lines in communication with the condensation and separation device and configured to permit removal of the condensed hydrocarbon-containing product, the purified inert gas, and the vaporized nitrogen from the condensation and separation device; and
a further separation device connected to and disposed downstream of the condensation and separation device and configured to purify the condensed hydrocarbon-containing product originating from the condensation and separation device by separating off dissolved gases.

29. The apparatus of claim 28, further comprising at least one dryer and at least one compressor in communication with and disposed, in a direction of process flow, between the residual monomer separation unit and the condensation and separation device.

30. The apparatus of claim 28, further comprising at least one heat exchanger in communication with and disposed down stream of said condensation and separation device and configured to heat the purified inert gas.

31. The apparatus of claim 30, wherein the at least one heat exchanger is a counterflow heat exchanger, configured to permit the flow there through of each of the hydrocarbon-containing inert gas that is to be separated and the purified inert gas separated therefrom, and in which the hydrocarbon-containing inert gas that is to be separated is cooled by the purified inert gas being heated.

32. The apparatus of claim 28, further comprising at least one heat exchanger in communication with the condensation and separation device and configured to heat nitrogen that has been vaporized in the condensation and separation device.

33. The apparatus of claim 32, wherein the at least one heat exchanger is a counterflow heat exchanger, configured to permit the flow there through of each of the hydrocarbon-containing inert gas that is to be separated and the vaporized nitrogen, and in which the hydrocarbon-containing inert gas that is to be separated is cooled by the vaporized nitrogen being heated.

34. The apparatus of claim 28, further comprising at least one heat exchanger in communication with the condensation and separation device and configured to heat the condensed hydrocarbon-containing product.

35. The apparatus of claim 34, wherein the at least one heat exchanger is a counterflow heat exchanger configured to permit the flow there through of each of the hydrocarbon-containing inert gas that is to be separated and the condensed hydrocarbon-containing product, and in which the hydrocarbon-containing inert gas that is to be separated may be cooled by the condensed hydrocarbon-containing product being heated.
